(19) United States
(12) Patent Application Publication
(10) Pub. No.: US 2026/0263123 A1
(43) Pub. Date: Sep. 10, 2026

(54) FEMORAL NECK METAL TRACK SLIDING SUPPORT LAG SCREW

(71) Applicant: Zhijun PAN, Yulin (CN)

(72) Inventors: Jingxin PAN, Yulin (CN); Yanming MA, Yulin (CN); Jianrui WANG, Yulin (CN); Zhijun PAN, Yulin (CN)

(73) Assignee: Zhijun PAN, Yulin (CN)

(21) Appl. No.: 19/466,439

(22) Filed: Feb. 1, 2026

(30) Foreign Application Priority Data
Feb. 24, 2025 (CN) 2025102004486

(51) Int. Cl. A61B 17/72; A61B 17/74

(52) U.S. Cl. CPC A61B 17/7241; A61B 17/7225; A61B 17/74

(57) ABSTRACT

Disclosed is a femoral neck sliding support lag screw system. An intramedullary main nail is disposed in a femoral trochanter region, and combined locking screws are inclinedly interlocked with the main nail to form an angularly stable structure. Each combined locking screw includes a sliding compression lag locking screw and a non-sliding support locking screw. A femoral neck fracture is fixed by two sliding compression lag locking screws, each supported at a middle-to-lower portion by the non-sliding support locking screw. The structure increases lag screw strength, reduces vertical toggle, and eliminates jamming after screw shaft lifting, improving sliding back-out capability and avoiding a Z-effect. A support platform and slope restrain displacement or deflection of the sliding compression lag locking screw, forming an angularly stable sliding track to achieve transverse stability and longitudinal compression.

### CROSS-REFERENCE TO RELATED APPLICATIONS

The application claims priority to Chinese patent application No. 2025102004486, filed on Feb. 24, 2025, the entire contents of which are incorporated herein by reference.

### TECHNICAL FIELD

The present disclosure relates to the technical field of medical devices, and more particularly to a femoral neck metal track sliding support lag screw.

### BACKGROUND

For a femoral neck fracture, since most of a blood supply to a femoral head is compromised, nonunion or femoral head necrosis is prone to occur. Also, since an effective fixation force arm of the femoral head is very short, and a weight-bearing line greatly deviates from a femoral shaft longitudinal axis, shear stress increases, and effective fixation is difficult. A mainstream fixation manner employs three cannulated compression screws for fixation. The cannulated screws possess good initial compression and subsequent sliding compression, but lack angular stability. Actual angular stability can only rely on a bony support force from mutual interdigitation at a fracture interface to be achieved. If osteoporosis, a comminuted fracture, or an excessive Pauwels angle is encountered, failure occurs easily. Meanwhile, screw placement requires sticking closely to a bone cortex around a femoral neck, and an operation difficulty is relatively large under conditions where navigation is absent. Fixation with a reconstruction nail possesses angular stability, but dyssynchronous sliding of two femoral neck locking screws causes a “Z-effect,” which is prone to failure. The FNS fixation system designed in the United States possesses both sliding compression and angular stability. However, a head screw is too bulky, bone loss inside the femoral head is excessive, damage to the blood supply is relatively large, a necrosis rate is relatively high, and absorption shortening is relatively large. Meanwhile, an anti-rotation capability of single-screw dominant fixation is relatively poor.

In view of this, how to combine biological characteristics of the femoral neck fracture to develop and design a new orthopedic internal fixation device for the femoral neck fracture to address the challenge of the femoral neck fracture becomes particularly important.

### SUMMARY

In view of deficiencies existing in the above prior art, the present disclosure aims to provide a femoral neck metal track sliding support lag screw. A sliding support track having metal angular stability is created for a lag screw inside a void femoral neck. Transverse stability and continuous longitudinal compression of a fracture are achieved by combined use with a trackless lag screw. The femoral neck metal track sliding support lag screw is provided with two sets of double-arm single-head combined locking screws parallel to each other and inclinedly connected to an intramedullary main nail. A femoral neck fracture is pressurized and fixed by two sliding compression lag locking screws. Support and stabilization corresponding to the sliding compression lag locking screw are respectively achieved utilizing two non-sliding support locking screws. Displacement of the sliding compression lag locking screw or jamming with the intramedullary main nail preventing backing out is effectively prevented. The femoral neck fracture is enabled to be pressurized and possess angular stability. A lower part of a middle section of the sliding compression lag locking screw is abutted against and supported utilizing the non-sliding support locking screw. Not only is a fulcrum of a lag locking screw moved medially to increase strength thereof, but also a middle part of the lag locking screw is lifted upward. A jamming phenomenon between the locking screw and the intramedullary main nail is eliminated, thereby eliminating a “Z-effect.” Also, a sliding track of the lag locking screw is extended inwardly by 40 mm, friction therebetween is reduced, sliding is made smoother, and screw backing out is made easier, thereby better addressing problems existing in the background section.

The present disclosure achieves the above technical objectives through the following technical solutions:

A femoral neck metal track sliding support lag screw includes an intramedullary main nail, and two sets of double-arm single-head combined locking screws interlocked with the intramedullary main nail. The intramedullary main nail is disposed within a femoral trochanter region. The two sets of double-arm single-head combined locking screws are parallel to each other, and the two sets of double-arm single-head combined locking screws are both inclinedly connected to the intramedullary main nail. A femoral neck fracture is fixed by the two sets of double-arm single-head combined locking screws.

As a further preferred solution of the above technical solution: each set of the double-arm single-head combined locking screws includes a sliding compression lag locking screw and a non-sliding support locking screw. Two parallel sliding compression lag locking screws pass through two first nail holes on the intramedullary main nail. Two parallel non-sliding support locking screws pass through two second nail holes on the intramedullary main nail. A top end of each support locking screw abuts against a location below an adjacent sliding compression lag locking screw.

To achieve the above objectives, further, an axial angle α1 between the sliding compression lag locking screw and the intramedullary main nail is 45°, and an axial angle α2 between the non-sliding support locking screw and the intramedullary main nail is 42.5°.

A further preferred solution is: a diameter of the sliding compression lag locking screw is 6.5 mm to 6.9 mm. A tail end thereof is provided with a self-tapping thread having a length of 15 mm to 20 mm.

A further preferred solution is: a spacing between the two sliding compression lag locking screws is 1.5 to 2 times a diameter of the sliding compression lag locking screw itself.

Based on the above technical solutions, further, a diameter of the non-sliding support locking screw is 6.5 mm. The non-sliding support locking screw is provided with an external thread. The non-sliding support locking screw is connected with the second nail hole through the external thread.

A further preferred solution is: a top end of the support locking screw is provided with a support part. The sliding compression lag locking screw is supported by the support part.

Preferably, the support part includes an arc-shaped support platform and a support slope. The arc-shaped support platform is opened at the top end of the non-sliding support locking screw. The support slope is located between the external thread and the arc-shaped support platform. A depth of the support slope gradually increases from a middle section to a tail part of the non-sliding support locking screw. A terminal of the support slope is connected to the arc-shaped support platform.

More preferably, a length of the arc-shaped support platform is 5 mm, and a depression depth of the arc-shaped support platform is 2.5 mm. An upper end of the arc-shaped support platform is an arc-shaped concave surface. A depth of the arc-shaped concave surface gradually deepens from near a head end of the non-sliding support locking screw to away from the head end of the non-sliding support locking screw.

Compared with the prior art, the present disclosure has the following beneficial effects:
1. The present disclosure is provided with two sets of double-arm single-head combined locking screws. The double-arm single-head combined locking screw includes the sliding compression lag locking screw and the non-sliding support locking screw. The two sliding compression lag locking screws therein are parallel to each other and a distance therebetween is 1.5 to 2 times the diameter of the sliding compression lag locking screw. The femoral neck fracture is pressurized and fixed utilizing the two sliding compression lag locking screws. Not only is the fracture continuously slid and pressurized, but also stability of a metal support angle is maintained. Abutment and support are performed on the lower part of the middle section of the sliding compression lag locking screw utilizing the non-sliding support locking screw. Not only is the fulcrum of the lag locking screw moved medially to increase strength thereof, but also a sliding track between the intramedullary main nail and the lag screw is extended, friction therebetween is reduced, sliding is made smoother, and backing out of the screw is made easier. The “Z-effect” existing during fixation of existing devices is eliminated.
2. In the present disclosure, the support part is provided at the tail end of the non-sliding support locking screw. The support part includes the arc-shaped support platform and the support slope. A cylindrical side surface where the non-sliding support locking screw originally contacts the sliding compression lag locking screw is designed in a form of combining the arc-shaped concave surface and an inclined plane. After the non-sliding support locking screw is rotated into position, the non-sliding support locking screw is supported on a side wall of the sliding compression lag locking screw in a semi-wrapping manner through the arc-shaped concave surface. Friction between the arc-shaped support platform and the sliding compression lag locking screw is increased, preventing relative displacement between the sliding compression lag locking screw and the non-sliding support locking screw. Meanwhile, a contact area with the sliding compression lag locking screw is increased utilizing the support slope, further enhancing stability of support of the non-sliding support locking screw for the sliding compression lag locking screw.
3. The structure of the present disclosure is simple and convenient to operate, and has a good fixation effect on a femoral fracture end. In actual use, the present disclosure is processed into various specifications according to clinical needs, meeting diverse needs of body characteristics of different patients, and improving universal applicability in clinical practice.

### BRIEF DESCRIPTION OF DRAWINGS

To more clearly describe technical solutions in embodiments of the present disclosure or in the prior art, drawings required to be used in the description of the embodiments or the prior art will be briefly introduced below.

FIG. 1 is a schematic diagram of an overall structure of a femoral neck metal track sliding support lag screw of the present disclosure.

FIG. 2 is an enlarged schematic diagram of a structure of the double-arm single-head combined locking screw in FIG. 1 of the present disclosure.

FIG. 3 is an enlarged schematic diagram of a partial structure of the double-arm single-head combined locking screw of the present disclosure.

FIG. 4 is a perspective view of the partial structure of the double-arm single-head combined locking screw of the present disclosure.

FIG. 5 is a sectional view of an intramedullary main nail of the present disclosure.

FIG. 6 is a schematic diagram of a structure of a sliding compression lag locking screw of the present disclosure.

FIG. 7 is a schematic diagram of a structure of a non-sliding support locking screw of the present disclosure.

FIG. 8 is a side view of the non-sliding support locking screw of the present disclosure.

FIG. 9 is a local enlarged schematic diagram of the non-sliding support locking screw of the present disclosure.

FIG. 10 is a front view of a shallower depression end of an arc-shaped support platform of the present disclosure.

FIG. 11 is a front view of a deeper depression end of the arc-shaped support platform of the present disclosure.

FIG. 12 is a first physical view of the non-sliding support locking screw of the present disclosure.

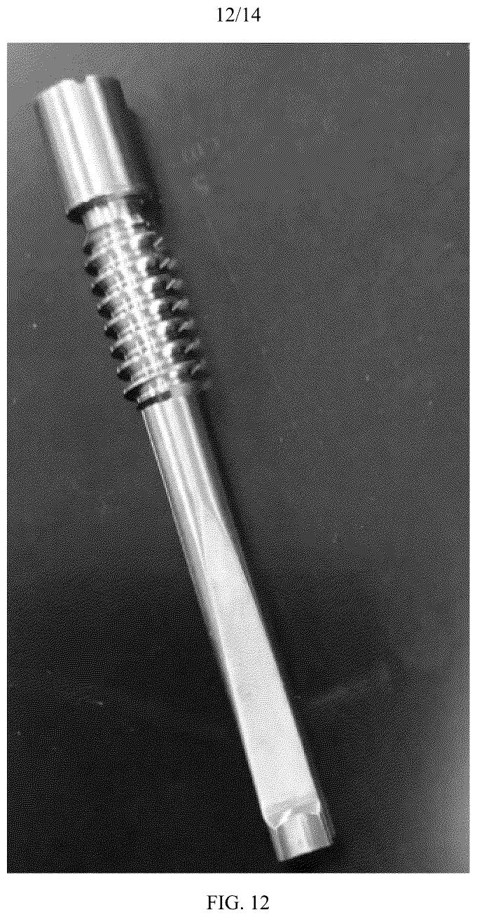

FIG. 13 is a second physical view of the non-sliding support locking screw of the present disclosure.

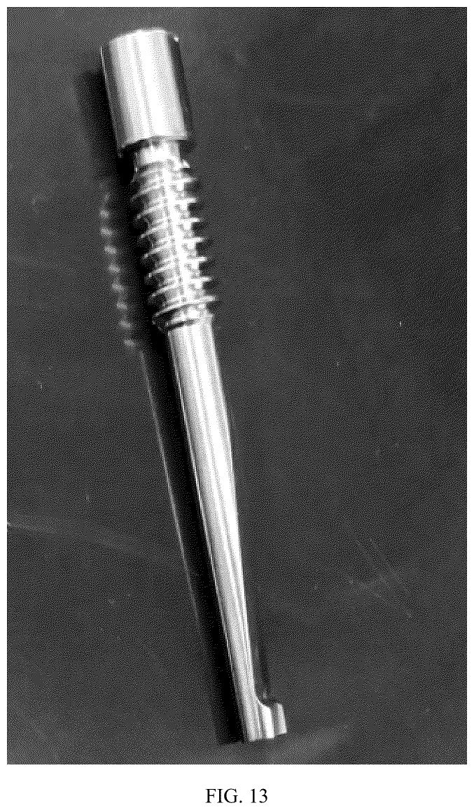

FIG. 14 is an X-ray image of a prior art operation in the background of the present disclosure.

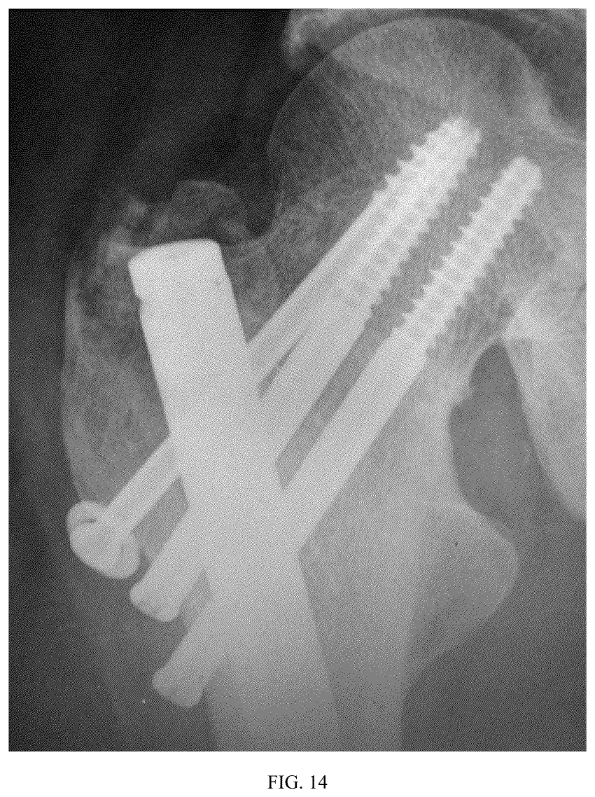

In the drawings: 1 intramedullary main nail; 11 first nail hole; 12 second nail hole; 2 double-arm single-head combined locking screw; 21 sliding compression lag locking screw; 211 bone self-tapping thread; 22 non-sliding support locking screw; 221 external thread; 222 arc-shaped support platform; 223 support slope.

### DETAILED DESCRIPTION OF THE EMBODIMENTS

To make objectives, technical solutions, and advantages of embodiments of the present disclosure clearer, technical solutions in the embodiments of the present disclosure will be clearly and completely described below with reference to drawings in the embodiments of the present disclosure. Obviously, the described embodiments are a part of the embodiments of the present disclosure, rather than all of the embodiments.

In the description of the present disclosure, it needs to be noted that orientation or position relationships indicated by terms “center,” “upper,” “lower,” “left,” “right,” “vertical,” “horizontal,” “inner,” “outer,” and the like are based on orientation or position relationships shown in the drawings, are merely for facilitating description of the present disclosure and simplifying the description, rather than indicating or implying that a referred device or element must have a specific orientation, be constructed and operated in a specific orientation, and therefore cannot be construed as a limitation on the present disclosure. Additionally, terms “first,” “second,” and the like are only used for description purposes, and cannot be construed as indicating or implying relative importance.

### Embodiment 1

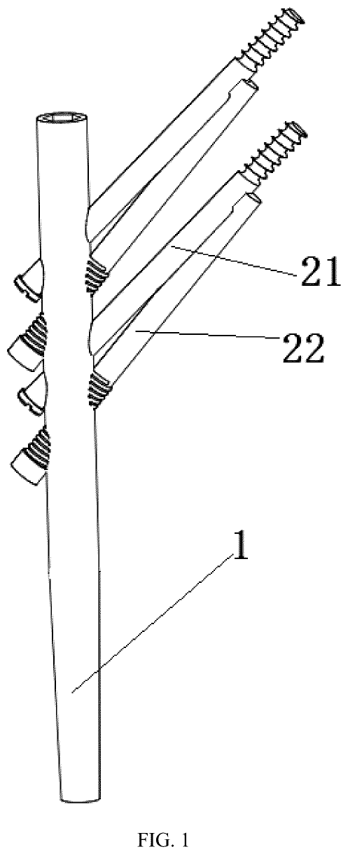

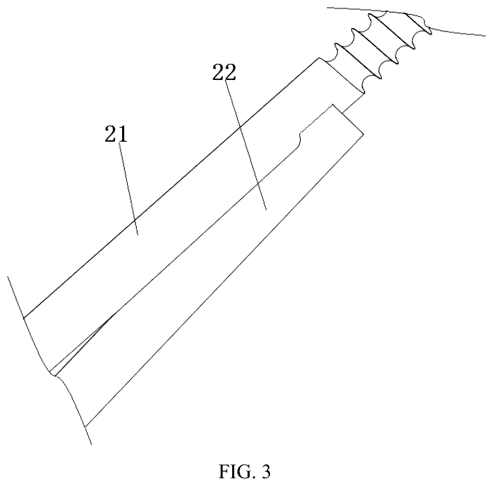

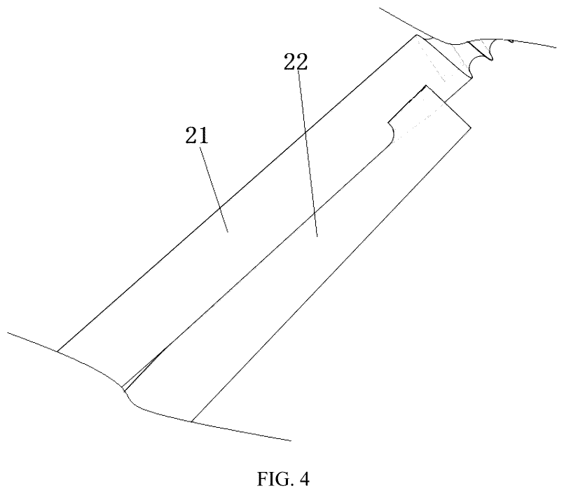

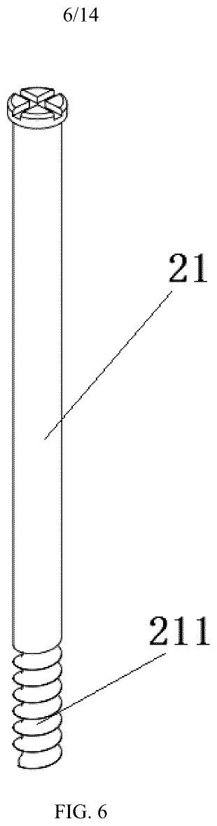

Referring to FIGS. 1 to 6, provided in the present disclosure is a femoral neck metal track sliding support lag screw, including an intramedullary main nail 1 and a double-arm single-head combined locking screw 2. The intramedullary main nail 1 is disposed within a femoral trochanter region. Two sets of double-arm single-head combined locking screws 2 are provided. The two sets of double-arm single-head combined locking screws 2 are parallel to each other and are both inclinedly connected to the intramedullary main nail 1. A fracture site of a proximal femur is fixed by the two sets of double-arm single-head combined locking screws 2.

Specifically, a head end of the intramedullary main nail 1 is provided with a countersunk inner hexagon groove. A length of the intramedullary main nail 1 is 180 mm. The intramedullary main nail 1 is composed of a columnar structure at an upper end and a tapered structure at a lower end. The columnar structure and the tapered structure transition smoothly and are integrally formed. A length of the columnar structure is 120 mm, and a diameter thereof is 13 mm. A length of the tapered structure is 60 mm, and a diameter of a terminal thereof is 9 mm to 10 mm.

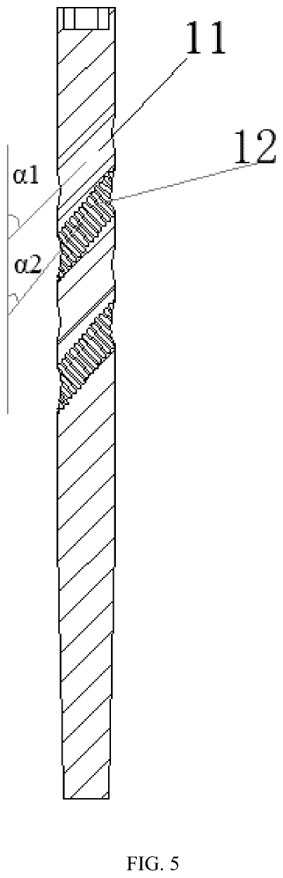

As shown in FIG. 5, first nail holes 11 and second nail holes 12 are sequentially opened on the intramedullary main nail 1 from top to bottom. There are two first nail holes 11 and two second nail holes 12. The two first nail holes 11 are parallel to each other, the two second nail holes 12 are parallel to each other, and the two first nail holes 11 and the two second nail holes 12 are distributed alternately.

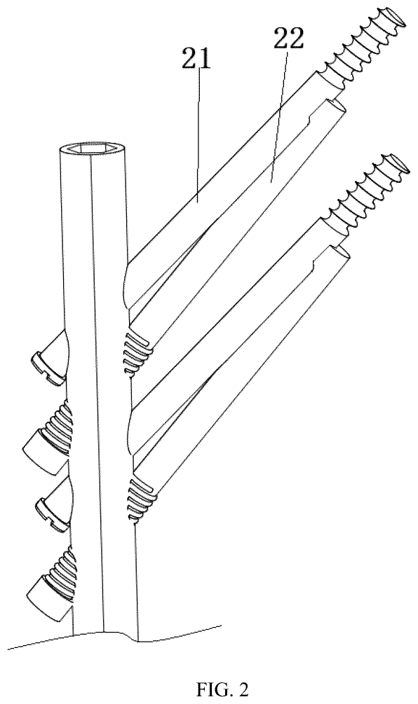

As shown in FIGS. 2 to 4, each set of the double-arm single-head combined locking screws 2 includes a sliding compression lag locking screw 21 and a non-sliding support locking screw 22. The non-sliding support locking screw 22 is located below the sliding compression lag locking screw 21. The sliding compression lag locking screw 21 is connected to the intramedullary main nail 1 through the first nail hole 11. An intersection of the sliding compression lag locking screw 21 and the intramedullary main nail 1 is a sliding connection, and screw backing out is possible. The non-sliding support locking screw 22 is connected to the intramedullary main nail 1 through the second nail hole 12. An intersection part of the non-sliding support locking screw 22 and the intramedullary main nail 1 is a threaded connection, and screw backing out does not occur. A top end portion of the non-sliding support locking screw 22 abuts against a location below the sliding compression lag locking screw 21, exerting a support and limiting effect on the sliding compression lag locking screw 21.

In this embodiment, a tail part of the sliding compression lag locking screw 21 is a Phillips screw. A diameter of the sliding compression lag locking screw 21 is 6.5 mm to 6.9 mm, and a length thereof is 85 mm to 100 mm. A bone self-tapping thread 211 having a length of 15 mm to 20 mm is provided at a top end of the sliding compression lag locking screw 21. A spacing between the two sliding compression lag locking screws 21 is 1.5 to 2 times the diameter of the sliding compression lag locking screw 21. After the sliding compression lag locking screw 21 passes through the first nail hole 11, an axial angle α1 formed with the intramedullary main nail 1 is 45°.

In this embodiment, a tail end of the non-sliding support locking screw 22 is provided with a countersunk inner hexagon groove. A diameter of the non-sliding support locking screw 22 is 6.5 mm, and a length thereof is of two types: 5.3 mm and 6.3 mm. The non-sliding support locking screw 22 is provided with an external thread 221. The external thread 221 is opened near a tail part thereof. A length of the external thread 221 is 27.5 mm. The external thread 221 cooperates with an internal thread in the second nail hole 12. The non-sliding support locking screw 22 is connected to the second nail hole 12 through the external thread 221. After the non-sliding support locking screw 22 passes through the second nail hole 12, an axial angle α2 formed with the intramedullary main nail 1 is 42.5°. The inner hexagon groove of the non-sliding support locking screw 22 is provided to facilitate fine adjustment of a position of the non-sliding support locking screw 22 by a surgeon using a screwdriver, ensuring effective support of the non-sliding support locking screw 22 for the sliding compression lag locking screw 21.

### Embodiment 2

On the basis of the above Embodiment 1, to better prevent the sliding compression lag locking screw 21 from undergoing displacement and angular rotation, the following further design is performed on a configuration of the non-sliding support locking screw 22.

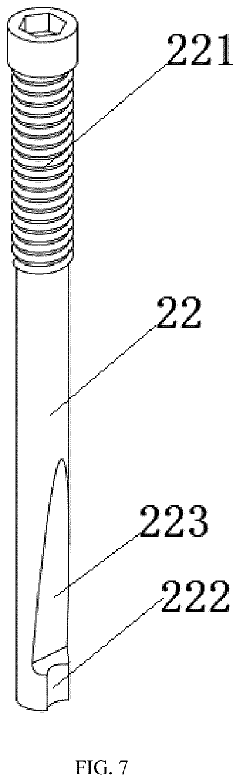

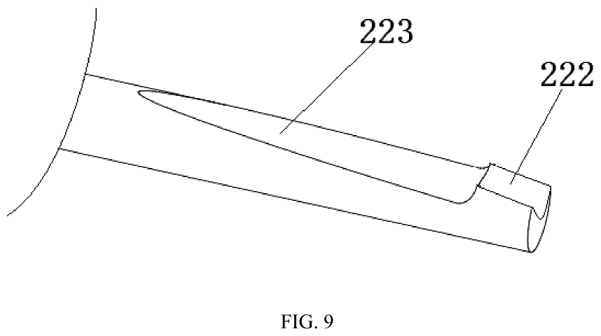

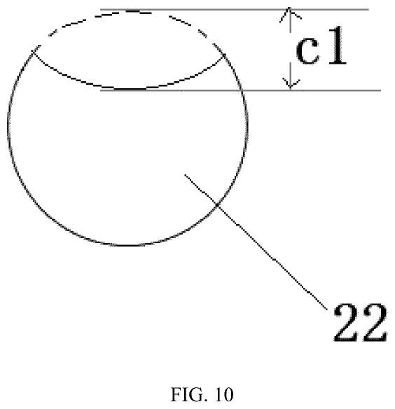

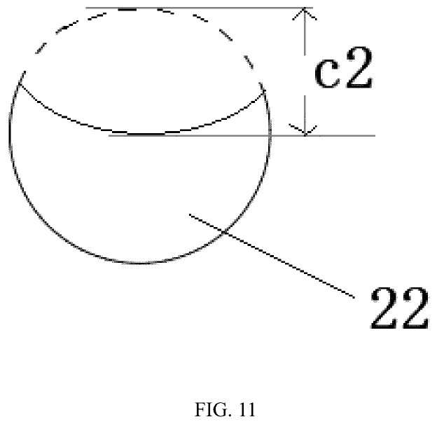

As shown in FIGS. 7 to 11, the top end of the non-sliding support locking screw 22 is provided with a support part. The support part includes an arc-shaped support platform 222 and a support slope 223. The arc-shaped support platform 222 and the support slope 223 are both formed by cutting on the non-sliding support locking screw 22. Specifically, the arc-shaped support platform 222 is opened at the top end of the non-sliding support locking screw 22. An upper end of the arc-shaped support platform 222 is an arc-shaped concave surface, and a depth of the arc-shaped concave surface gradually deepens from near a head end of the non-sliding support locking screw 22 toward away from the head end of the non-sliding support locking screw 22. That is, a cross-section of the arc-shaped support platform 222 assumes a “crescent” shape that gradually decreases (refer to FIG. 10 and FIG. 11). The support slope 223 is an inclined plane. The support slope 223 is located between the external thread 221 and the arc-shaped support platform 222. A depth of the support slope 223 gradually increases from a middle section to a tail part of the non-sliding support locking screw 22. A terminal of the support slope 223 extends to connect with the arc-shaped support platform 222. Meanwhile, a height of the terminal of the support slope 223 does not exceed a lowest support point of the arc-shaped support platform 222.

When the non-sliding support locking screw 22 is rotated into position, the non-sliding support locking screw 22 is supported on a bottom side wall of the sliding compression lag locking screw 21 in a semi-wrapping manner through the arc-shaped concave surface. Contact between the arc-shaped support platform 222 and the sliding compression lag locking screw 21 is increased, which is capable of effectively preventing relative displacement between the sliding compression lag locking screw 21 and the non-sliding support locking screw 22. Meanwhile, a contact area with the sliding compression lag locking screw 21 is further increased utilizing the support slope 223, enhancing stability of support of the non-sliding support locking screw 22 for the sliding compression lag locking screw 21.

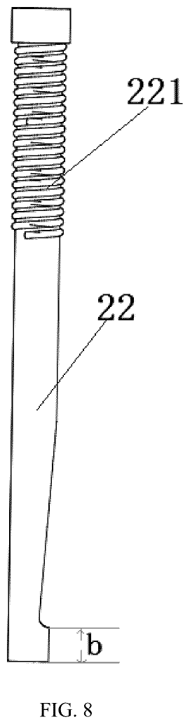

In this embodiment, as shown in FIGS. 8 and 10 to 11, a length b of the arc-shaped support platform 222 is 5 mm. A depression depth of the arc-shaped support platform 222 extends from 2.5 mm near the head end c1 of the non-sliding support locking screw 22 to 3 mm away from the head end c2 of the non-sliding support locking screw 22.

### Embodiment 3

When the femoral neck metal track sliding support lag screw of the present disclosure is applied in clinical practice:

According to a clinical treatment solution, a patient undergoes closed reduction and temporary fixation in emergency (or sub-emergency). After anesthesia, the patient is placed on a traction table for reduction. After AP and lateral fluoroscopy determine that a fracture is generally reduced, three Kirschner wires having a diameter of 2.5 mm are driven into a femoral neck for temporary fixation. Simultaneously, ipsilateral tibial tubercle skeletal traction immobilization is performed. A traction weight is 6 kg. After traction for 7 to 9 days, definitive surgery treatment is then performed. Since closed immobilization enables original traumatic inflammation to fully subside, fracture healing ability is greatly increased, and an extent of absorption/shortening of a fracture end is greatly reduced (or even eliminated). After traction for one week, open reduction for a patient with poor surgical reduction may also produce a secondary traumatic healing response, increasing the fracture healing ability. Bone grafting may also be performed to increase the healing ability when necessary.

During surgery, the patient is placed on the traction table again. A 5 cm to 6 cm longitudinal incision is made on skin above the femoral trochanter region to fully expose a tip of the trochanter. First, a guide pin is inserted. Fluoroscopy confirms that the guide pin is placed at an appropriate position in a medullary cavity. Then, proximal femur reaming is performed. Before reaming, the Kirschner wires obstructing the reaming are removed. Additionally, a Kirschner wire is driven at an appropriate position to maintain reduction. The intramedullary main nail is inserted to an appropriate depth. Beside the intramedullary main nail (mostly on an anterior side), 1 to 2 trackless cannulated screws having a diameter of 7.3 mm are driven as perpendicular to a fracture line as possible for compression fixation. After the fracture line completely disappears, two non-sliding support locking screws 22 are driven. Finally, two sliding compression lag locking screws 21 are driven. For a patient without anatomical reduction or with poor fracture prognosis, open reduction and bone grafting are immediately performed to increase the fracture healing ability.

It needs to be noted that, in clinical actual application, 1 to 2 cannulated screws of 7.3 mm are placed outside the intramedullary main nail for compression fixation. A reconstruction nail angular stability concept is combined with a cannulated screw compression concept. Not only are multiple small-diameter screw fixation advantages of the cannulated screw retained, but also “bony support angular stability” is successfully transformed into “metal support angular stability.” Bone resorption caused by micromotion at the fracture site may be reduced (meanwhile, delayed surgery increases bone end growth ability, further reducing bone end absorption). Shortening from screw back-out is reduced. Moreover, strict cortical contact fixation of a pure cannulated screw is not required, reducing intraoperative fluoroscopic positioning difficulty and facilitating conduct of clinical work in primary hospitals.

The basic principle, main features, and advantages of the present disclosure are shown and described above. Persons skilled in the art should understand that the present disclosure is not limited by the above embodiments. The above embodiments and the description only describe the principle of the present disclosure. Various changes and modifications may also occur to the present disclosure without departing from the spirit and scope of the present disclosure. These changes and modifications all fall within the scope of the present disclosure claimed for protection. The scope of protection of the present disclosure is defined by the appended claims and equivalents thereof.

What is claimed is:

1. A femoral neck metal track sliding support lag screw, comprising: an intramedullary main nail 1; and two sets of double-arm single-head combined locking screws 2 connected to the intramedullary main nail 1, wherein the intramedullary main nail 1 is disposed within a femoral trochanter region; wherein the two sets of double-arm single-head combined locking screws 2 are parallel to each other; wherein each of the two sets of double-arm single-head combined locking screws 2 is inclinedly connected to the intramedullary main nail 1; and wherein a femoral neck fracture is fixed by the two sets of double-arm single-head combined locking screws 2.

2. The femoral neck metal track sliding support lag screw according to claim 1, wherein each set of the double-arm single-head combined locking screws 2 comprises a sliding compression lag locking screw 21 and a non-sliding support locking screw 22; wherein two parallel sliding compression lag locking screws 21 pass through two first nail holes 11 on the intramedullary main nail 1; wherein two parallel non-sliding support locking screws 22 pass through two second nail holes 12 on the intramedullary main nail 1; and wherein a top end of each non-sliding support locking screw 22 abuts against a location below an adjacent sliding compression lag locking screw 21.

3. The femoral neck metal track sliding support lag screw according to claim 2, wherein an axial angle α1 between the sliding compression lag locking screw 21 and the intramedullary main nail 1 is 45°; and wherein an axial angle α2 between the non-sliding support locking screw 22 and the intramedullary main nail 1 is 42.5°.

4. The femoral neck metal track sliding support lag screw according to claim 3, wherein a diameter of the sliding compression lag locking screw 21 is 6.5 mm to 6.9 mm; and wherein a top end of the sliding compression lag locking screw 21 is provided with a bone self-tapping thread 211 having a length of 15 mm to 20 mm.

5. The femoral neck metal track sliding support lag screw according to claim 4, wherein a spacing between the two sliding compression lag locking screws 21 is 1.5 to 2 times a diameter of the sliding compression lag locking screw 21.

6. The femoral neck metal track sliding support lag screw according to claim 3, wherein a diameter of the non-sliding support locking screw 22 is 6.5 mm; wherein the non-sliding support locking screw 22 is provided with an external thread 221; and
wherein the non-sliding support locking screw 22 is connected to the second nail hole 12 through the external thread 221.

7. The femoral neck metal track sliding support lag screw according to claim 6, wherein a top end of the non-sliding support locking screw 22 is provided with a support part; and wherein the sliding compression lag locking screw 21 is supported by the support part.

8. The femoral neck metal track sliding support lag screw according to claim 7, wherein the support part comprises an arc-shaped support platform 222 and a support slope 223; wherein the arc-shaped support platform 222 is opened at the top end of the non-sliding support locking screw 22; wherein the support slope 223 is located between the external thread 221 and the arc-shaped support platform 222; wherein a depth of the support slope 223 gradually increases from a middle section to a tail part of the non-sliding support locking screw 22; and wherein a terminal of the support slope 223 is connected to the arc-shaped support platform 222.

9. The femoral neck metal track sliding support lag screw according to claim 8, wherein a length of the arc-shaped support platform 222 is 5 mm; and wherein a depression depth of the arc-shaped support platform 222 is 2.5 mm to 3 mm.

10. The femoral neck metal track sliding support lag screw according to claim 9, wherein an upper end of the arc-shaped support platform 222 is an arc-shaped concave surface; and wherein a depth of the arc-shaped concave surface gradually deepens from near a head end of the non-sliding support locking screw 22 to away from the head end of the non-sliding support locking screw 22.
